(19) United States
(12) Patent Application Publication
(10) Pub. No.: US 2021/0089121 A1
(43) Pub. Date: Mar. 25, 2021

(54) USING SPATIAL INFORMATION FOR DYNAMIC DOMINANT EYE SHIFTS

(71) Applicant: Tobii AB, Danderyd (SE)

(72) Inventor: Denny Rönngren, Danderyd (SE)

(73) Assignee: Tobii AB, Danderyd (SE)

(21) Appl. No.: 16/578,734

(22) Filed: Sep. 23, 2019

(51) Int. Cl. G06F 3/01; A61B 3/00; A61B 3/113; G06F 3/0484

(52) U.S. Cl. CPC G06F 3/013; A61B 3/0025; G06F 3/0484; A61B 3/0033; A61B 3/113

(57) ABSTRACT

Systems and methods for gaze origin selection based on dominant eye shift of a user. Systems and methods described herein include eye tracking devices and computing devices. The first eye is initially set as the dominant eye. A first gaze ray for the first eye and a second gaze ray for a second eye are determined by an eye tracking system. The first gaze ray intersects a first object and the second gaze ray intersects a second object within the field of view, at a first depth and a second depth respectively. A convergence depth of the first gaze ray and the second gaze ray is determined and compared against the first depth and the second depth. When a second offset between the second depth and the convergence depth is smaller than a first offset between the first depth and the convergence depth, the second eye is set as the dominant eye.

### TECHNICAL FIELD

The present disclosure generally relates to the field of eye tracking. In particular, the present disclosure relates to a method and system for gaze origin selection based on dynamic dominant eye shifts.

### BACKGROUND

Interaction with computing devices is a fundamental action in today's world. Computing devices, such as personal computers, tablets, and smartphones, are found throughout daily life. In addition, computing devices that are wearable, such as wearable headset devices (e.g., virtual reality (VR) headsets and augmented reality (AR) headsets), are becoming increasingly popular. The systems and methods for interacting with such devices define how they are used and what they are used for.

Advances in eye tracking technology have made it possible to interact with a computing device using a person's gaze information, such as the position on a display the user is gazing at. This information can be used for interaction solely, or in combination with a contact-based interaction technique (e.g., using a user input device, such as a keyboard, a mouse, a touch screen, or another input/output interface).

Previously proposed interaction techniques using gaze information can be found in U.S. Pat. No. 6,204,828, United States Patent Application Publication 2013/0169560, U.S. Pat. No. 7,113,170, United States Patent Application Publication 2014/0247232, and U.S. Pat. No. 9,619,020. The full specification of these patents and applications are herein incorporated by reference.

In computing devices, such as wearable devices, images of an eye may be generated and processed to detect the pupil and glints and, thereby, track the eye. In many implementations, a light source, such as one including a light emitting diode (LED), is powered on to illuminate the eye and for the imaging. In such devices and systems it is important to have accurate gaze estimation for proper tracking and functioning of the related computer systems. Typically both eyes of a user are tracked and the gaze origin for the gaze direction of the user is set as the midpoint between the users eyes. Further improvements are desired for more accurate gaze tracking based on data gathered from the eyes.

### SUMMARY

Various examples are described including systems, methods, and devices relating to gaze origin selection in eye tracking systems based on dynamic eye dominance shifting.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method in an eye tracking system for gaze origin selection based on dominant eye shift of a user, including receiving depth information of a first object and a second object displayed on a display device and receiving eye tracking data indicating a first gaze ray of a first eye of the user and a second gaze ray of a second eye of the user. The method further includes setting the first eye as a dominant eye. The method also includes determining that the first gaze ray intersects the first object at a first depth and determining that the second gaze ray intersects the second object at a second depth. The method includes determining a convergence depth at a convergence of the first gaze ray and the second gaze ray. The method then includes determining a first offset between the convergence depth and the first depth and determining a second offset between the convergence depth and the second depth. When the second offset is smaller than the first offset the method also includes setting the second eye as the dominant eye based on the second offset being smaller than the first offset. Other embodiments of this aspect include corresponding computer systems, apparatus, and computer programs recorded on one or more computer storage devices, each configured to perform the actions of the methods.

Another general aspect includes a computer system, including a non-transitory computer-readable medium storing instructions; and a processing device communicatively coupled to the non-transitory computer-readable medium, where the processing device is configured to execute the instructions and perform operations including receiving depth information of a first object and a second object displayed on a display device and receiving eye tracking data indicating a first gaze ray of a first eye of a user and a second gaze ray of a second eye of the user. The instructions further include setting the first eye as a dominant eye. The instructions also include determining that the first gaze ray intersects the first object at a first depth and that the second gaze ray intersects the second object at a second depth. The instructions then cause the computer system to determine a convergence depth at a convergence of the first gaze ray and the second gaze ray. The instructions then include determining a first offset between the convergence depth and the first depth and a second offset between the convergence depth and the second depth. The instructions cause the computer system to determine that the second offset is smaller than the first offset and set the second eye as the dominant eye based on the second offset being smaller than the first offset. Other embodiments of this aspect include corresponding computer systems, apparatus, and computer programs recorded on one or more computer storage devices, each configured to perform the actions of the methods.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 is a block diagram of one possible system for gaze origin selection based on dynamic dominant eye shifts, according to at least one example.

FIG. 2 illustrates a diagram illustrating an example of gaze rays for a first and a second eye converging on a first object with a first gaze ray initiated at a first eye which is dominant, according to at least one example.

FIG. 3 illustrates a diagram illustrating an example of the first gaze ray being obstructed by a first object and causing eye dominance to shift to a second eye, according to at least one example.

FIG. 4 illustrates a diagram illustrating an example of gaze rays for a first and a second eye intersecting a second object maintaining the second eye as a dominant eye after the first object no longer obstructs the first gaze ray, according to at least one example.

FIG. 5A illustrates an example of an image presented to a left eye of a user including a second object partially obscured by a first object, according to at least one example.

FIG. 5B illustrates an example of an image presented to a right eye of a user including the first object obscuring the second object, according to at least one example.

FIG. 6A illustrates an example of an image presented to a left eye of a user including a second object partially obscured by a first object and also including a mask for removing portions of the image obstructed by physical objects, according to at least one example.

FIG. 6B illustrates an example of an image presented to a right eye of a user including the first object obscuring the second object and also including a mask for removing portions of the image obstructed by physical objects, according to at least one example.

FIG. 7 is a flow diagram of one example method for gaze origin selection based on dynamic dominant eye shifts, according to at least one example.

FIG. 8 is a block diagram of an exemplary computer system capable of being used in at least some portion of the devices or systems of the present disclosure, or implementing at least some portion of the method of the present disclosure, according to at least one example.

In the appended figures, similar components and/or features may have the same numerical reference label. Further, various components of the same type may be distinguished by following the reference label by a letter that distinguishes among the similar components and/or features. If only the first numerical reference label is used in the specification, the description is applicable to any one of the similar components and/or features having the same first numerical reference label irrespective of the letter suffix.

### DETAILED DESCRIPTION

Embodiments for gaze origin selection based on dominant eye shift of a user are disclosed. A ray-casting algorithm projects a virtual ray along the gaze direction of each eye as measured or detected by an eye tracking device. In an example, a computing device receives depth information including a depth of a first object and a second object as well as eye tracking data indicating a gaze ray for each of a right eye and a left eye, the gaze ray originating at each eye and directed in the gaze direction of the eye. Each gaze ray is a vector that has a direction and an origin, the origin located at the pupil of the respective eye. One of the two eyes is initially set as a dominant eye. The gaze ray of each eye may intersect with the first object or the second object, respectively. This would typically result in inconsistent object selection, however, in this example, the computing device determines a convergence depth of the first gaze ray and the second gaze ray. The convergence depth describes a distance from a point in relation to a user's eyes to a point where the user is currently focusing. In examples described herein, the point in relation to the user's eyes is the dominant eye of the user, which may dynamically shift. The computing device then compares the depth of the first object and the second object to the convergence depth. If the convergence depth is within a predetermined offset threshold of the second object then the computing device will set whichever eye originates the gaze ray that intersects the second object as the dominant eye and the gaze origin for object selection. In one example, the gaze origin and gaze ray of the dominant eye is used for object selection based on ray-casting.

The technologies presented herein improve object selection based on ray-casting in eye tracking systems by comparing the convergence depth of gaze rays from each of the user's eyes to the depth of objects within the field of view and making a selection based on ray-casting from one of the user's eyes. The dominant eye of the user may dynamically shift based on proximity or movement of objects within the field of view. For example, a user may typically be right eye dominant, but may be viewing on object in a VR or AR environment that is totally or partially obscured from view of the right eye and therefore dynamically switch eye dominance to the left eye. In such cases, object selection based on ray-casting is improved by dynamically adjusting eye dominance of the user.

The ensuing description provides exemplary embodiments only and is not intended to limit the scope, applicability or configuration of the disclosure. Rather, the ensuing description of the exemplary embodiments will provide those skilled in the art with an enabling description for implementing one or more exemplary embodiments. It is understood that various changes may be made in the function and arrangement of elements without departing from the scope of the disclosure as set forth in the appended claims.

Specific details are given in the following description to provide a thorough understanding of the embodiments. However, it will be understood by one of ordinary skill in the art that the embodiments may be practiced without these specific details. For example, circuits, systems, networks, processes, and other elements in the present disclosure may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but could have additional steps not discussed or included in a figure. Furthermore, not all operations in any particularly described process may occur in all embodiments. A process may correspond to a method, a function, a procedure, a subroutine, a subprogram, etc. When a process corresponds to a function, its termination corresponds to a return of the function to the calling function or the main function. Any detail present in one discussed embodiment may or may not be present in other versions of that embodiment or other embodiments discussed herein.

The term “computer-readable medium” includes, but is not limited to portable or fixed storage devices, optical storage devices, wireless channels and various other mediums capable of storing, containing or carrying instruction(s) and/or data. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

Furthermore, embodiments presented herein may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

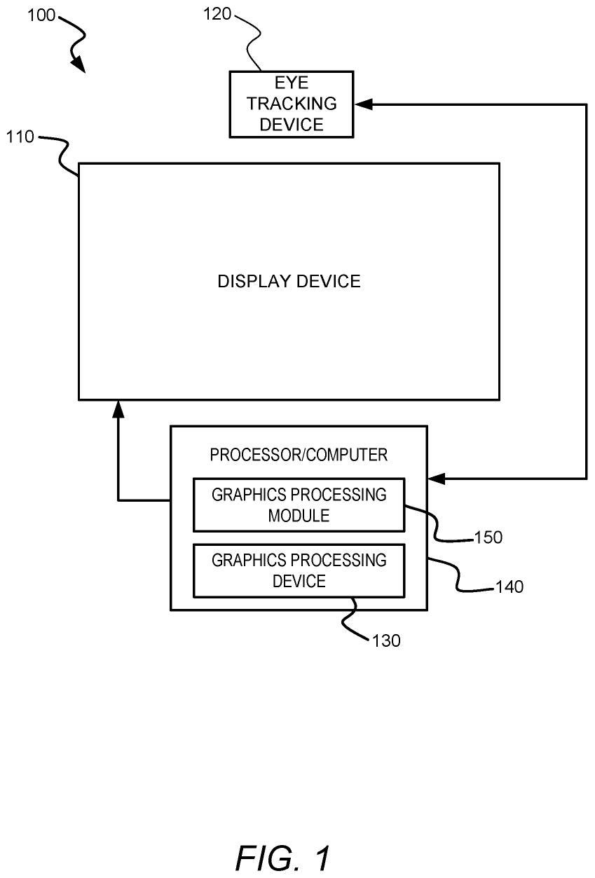

In some embodiments, and with reference to FIG. 1, a system 100 for object selection based on dynamic dominant eye shifts is provided. The system 100 may include an eye tracking device 120 and a graphics processing device 130. In some embodiments, the system 100 may also include a computing device 140 which communicates with, and controls, a graphics processing device 130. In some embodiments, any function of the graphics processing device 130 may be performed, in whole or in part, by a computing device 140. Part of the function of the graphics processing device 130 may also be performed by another computing device or on the cloud. Merely by way of example, the eye tracking device 120 may be provided integral to, or in addition to, computing device 140 having a graphics processing device 130 and a central processing unit, such as a personal computer (in some configurations, the graphics processing device 130 and the central processing unit are integrated).

In other embodiments, the eye tracking device 120 may be provided integral to, or in addition to, the computing device 140, such as a gaming console or other device having a graphics processing device 130 and a central processing unit. In other embodiments, the eye tracking device 120 may be provided integral to, or in addition to, a wearable headset such as a Virtual Reality (VR) or Augmented Reality (AR) or the like. Likewise, the display device 110 may also be a display device integral to a wearable headset and the computing device 140 may also be integral to the wearable headset. The system 100 can thus be a wearable headset with a head-mounted display (HMD). Thus, embodiments presented herein may be applied to the presentation of graphics in any number of possible devices and applications, including a video display, video games, video production and editing, video communications, computer-aided drafting, and design, etc.

The eye tracking device 120 may be for determining at least one of a gaze point of a user on a display device 110, or a change in the gaze point of the user on display device 110. Eye tracking devices and methods, sometimes referred to as gaze detection systems and methods include, for example, products produced and available from Tobii AB, and which operate by using infrared illumination and an image sensor to detect reflection from the eye of a user. An example of such a gaze detection system is described in U.S. Pat. No. 7,572,008, which is hereby incorporated by reference, for all purposes, as if fully set forth herein. Other alternative gaze detection systems may also be employed, regardless of the technology behind the gaze detection system. The eye tracking device 120 may employ its own processor or the processor of another device (i.e., the computing device 140) to interpret and process data received. When an eye tracking device is referred to herein, both possible methods of processing data are referred to.

In some embodiments, the graphics processing device 130 renders the computer-generated scene, such as three-dimensional (3D) scene, into images and send the rendered images to the display device 110 for display. The graphics processing device 130 may further include a graphics processing module 150 for processing the rendered images, such as combining high quality portion and low quality background image, or perform other post-processing operations on the rendered image. In some examples, the graphics processing module 150 also generates a depth map of objects within the 3D scene. In some examples, the graphics processing module 150 generates depth information for selectable objects or objects at or near a gaze ray of the user. In some embodiments, the graphics processing module 150 also performs the image re-projection mechanism before the rendered image is sent to the display device 110. The image rendering may also be performed by another computing device locally or over the cloud and then the rendered image is sent to the display device 110 through a wired or wireless connection. While in some embodiments a separate non-included or non-integrated display device will be controlled by the graphics processing device 130, other embodiments may include or integrate the display device 110.

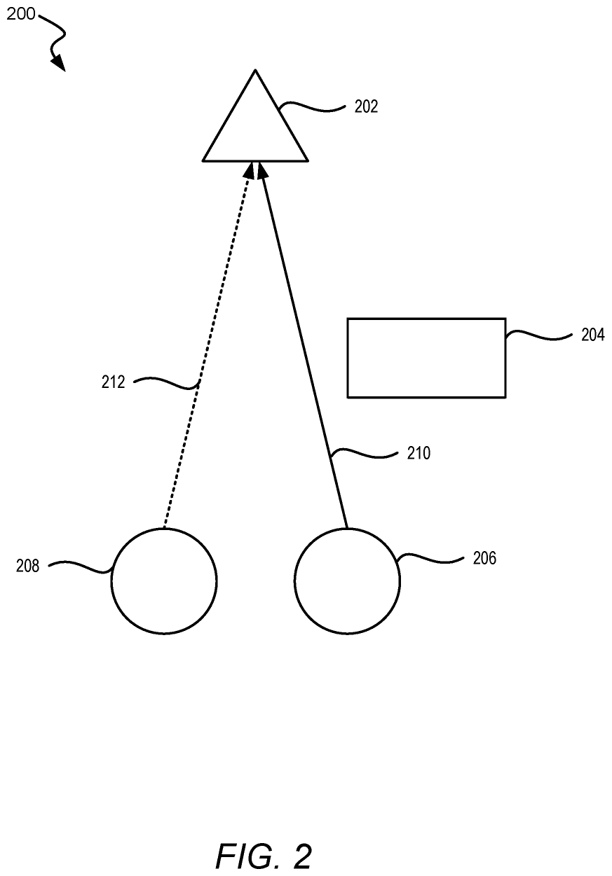

FIG. 2 depicts a diagram 200 illustrating a user's field of view while using an eye tracking system with rays identifying the gaze ray of each of the user's eyes. The right eye 206 is a first eye of the user and the left eye 208 is a second eye of the user in this example for purposes of illustration. In other examples the first eye and the second eye may be different than depicted and described herein, such as the first eye being the left eye 208 and the right eye 206 being the second eye. The right eye 206 is shown with a first gaze ray 210 extending from the pupil of the right eye 206 along an axis of the gaze ray of the right eye 206. The left eye is shown with a second gaze ray 212 extending from the pupil of the left eye 208 along an axis of the gaze ray of the left eye 208. The right eye 206 is the dominant eye of the user and therefore the first gaze ray 210 is used for object selection by ray-casting. The first gaze ray 210 serves as the ray used for ray-casting, originating at the pupil of the right eye 206. The first gaze ray 210 is depicted as a solid line to indicate that it originates at the dominant eye.

The first gaze ray 210 and the second gaze ray 212 intersect at a convergence depth where they intersect with a second object 202. The convergence depth may be determined based on an interpupillary distance or other methods known in the art. An example of such a gaze detection system is described in U.S. patent application Ser. No. 16/220,760, which is hereby incorporated by reference, for all purposes, as if fully set forth herein. The gaze convergence depth describes a distance from a point in relation to a user's eyes to a point where the user is currently focusing. The point in relation to the user's eyes could for example be a point halfway between the rotation centers of the user's eyes, could be one of either the left eye 208 or the right eye 206, or could be a point located between the right eye 206 and the left eye 208 besides the halfway point, such as biased towards a user's dominant eye. In one example, the point from which the gaze convergence depth is measured is the pupil center of the user's eyes. In an example, to determine the convergence depth, first, calibration data are determined in relation to an interpupillary distance between a pupil of a left eye 208 and a pupil of a right eye 206 of a user. Calibration data refer to data for calibrating a method to a specific user, a specific eye tracking system and/or other varying factors. For example, interpupillary distance for users focusing at a specific depth of field differs between users due to differing anatomy of the users. Furthermore, depending on the type of system and other factors, the length between a user's eyes and imaging devices used to capture images used in the method will differ. The calibration data required will generally differ depending on the gaze convergence function used. Determining calibration data may be realized by means of retrieval of general approximations or by means of performing measurements or a combination thereof. General approximations may be based on average data for human eyes and may depend on the specific application and/or system in which the method is implemented. Measurements performed may be in relation to the eyes of a specific user. Based on the determined calibration data, a gaze convergence function providing an approximate gaze convergence depth of the user based on a determined interpupillary distance of the user is determined. The gaze convergence function will generally differ between different application, e.g., due to different need for the accuracy needed for the approximation of the convergence depth and/or what the approximate gaze convergence depth should be used for. To determine a current interpupillary distance, one or more images of the left eye 208 and the right eye 206 of the user are then received from one or more imaging devices and the current interpupillary distance of the user is determined based on the one or more images. A current approximate gaze convergence depth is then determined based on the current interpupillary distance and the gaze convergence function.

One example gaze convergence function f(x) providing an approximation of a gaze convergence depth based on interpupillary distance (x) is shown below. If the eyes of a user are approximated with spheres with radius r and a shortest distance between the spheres x0 and distance between rotation centers of the spheres x∞, and the pupils approximated to be at the surfaces of the spheres and to point in direction towards each other when gaze convergence depth is 0 and parallel to each other when the convergence depth is ∞ then the distance between the pupils at convergence depth 0 is x0 and distance between the pupils at convergence depth ∞ is x∞. In this case the convergence depth is measured from a point halfway between the rotation centers of the eye balls. Furthermore, it can be derived that x∞=x0+2r. A function f(x) approximating the convergence depth for distances x between pupils (interpupillary distance) can be derived for x0≤x≤x∞ using the approximated geometry. The approximate function is

$$f ( x ) = \frac{\sqrt{r ( x - {x}_{0} ) - {( \frac{x - {x}_{0}}{2} )}^{2}}}{( 1 - \frac{x}{{x}_{0} + 2 r} )}$$

The function f(x) above relates to the case where the user is focusing at a point along a line starting from a point halfway between the rotation centers and being perpendicular to a line between the rotation centers. This assumption is reasonable in many cases as a user focusing a certain object will normally turn her/his head to achieve such a situation.

The depth of the second object 202 is known from the position information, either through the use of a depth map of virtual objects, or through the use of a depth sensor positioned on an eye tracking system and used in conjunction with an augmented reality system. A first object 204 is also present in the scene at a second depth in close proximity to the first gaze ray 210, in a first position, though not obstructing the view of the second object 202 by the right eye 206. The depth of the first object 204 is known from the position information, again either through the use of a depth sensor or depth map by the computing device.

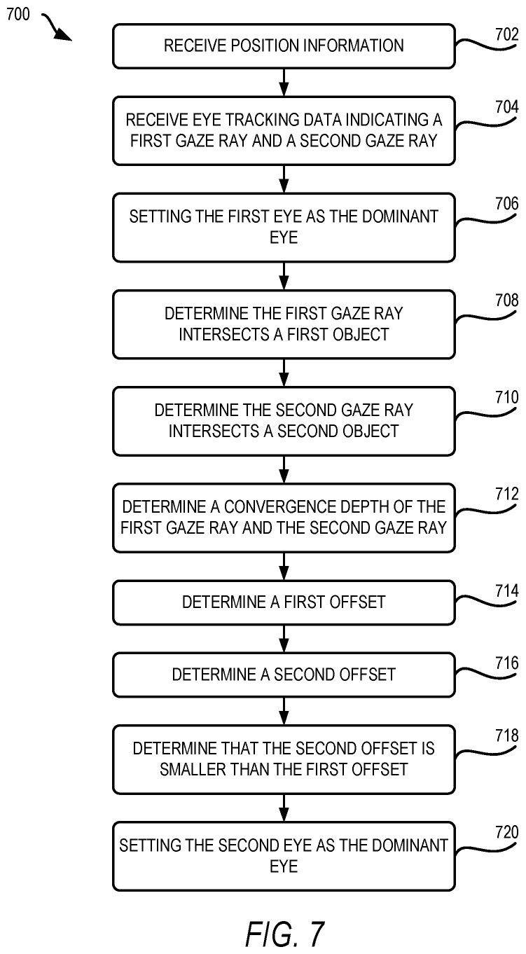

As described below, with respect to FIG. 7, the convergence depth is used to determine which gaze ray, and therefore which eye of a user is the dominant eye and should be relied upon for object selection.

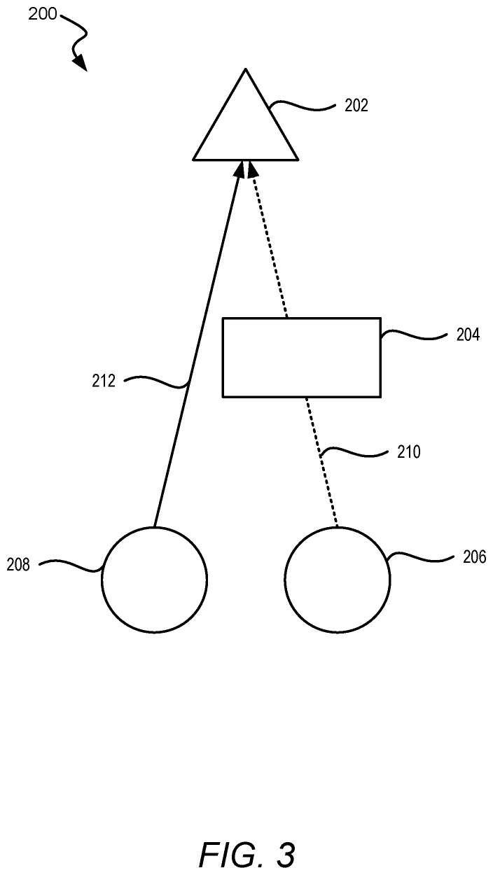

FIG. 3 shows the diagram 200 with the first object 204, at the second depth, in a second position, located between the right eye 206 and the second object 202, such that the first gaze ray 210 intersects the first object 204 before reaching the second object 202. The second gaze ray 212 has an unobstructed view of the second object 202. In this diagram 200, the second gaze ray 212 is a solid line representing it is the ray that will be used for object selection by ray-casting. If the first gaze ray 210 is used to select an object, the first object 204 would incorrectly be selected. Instead, eye dominance shifts such that the left eye 208 is the dominant eye and the second gaze ray 212 is used for object selection.

The transition of diagram 200 from FIG. 2 to FIG. 3 could be the result of the first object 204 moving from right to left, or alternatively may be the result of the user moving sideways towards the right. In one illustrative example, in a first person game enabled and operated by a user with an eye tracking device, the user may navigate through the virtual environment such that they are partially obscured by a wall by moving behind the wall and peeking around the edge of the wall. The wall is represented in FIG. 3 by the first object 204. In a first person game, the user may operate object selection based on the gaze ray of the dominant eye. In typical systems, the eye tracking system may incorrectly select the first object 204, while the present system is able to correctly identify that the first gaze ray 210 and the second gaze ray 212 converge at a convergence depth at or near the second object 202 and therefore select the second object 202 and not the first object 204.

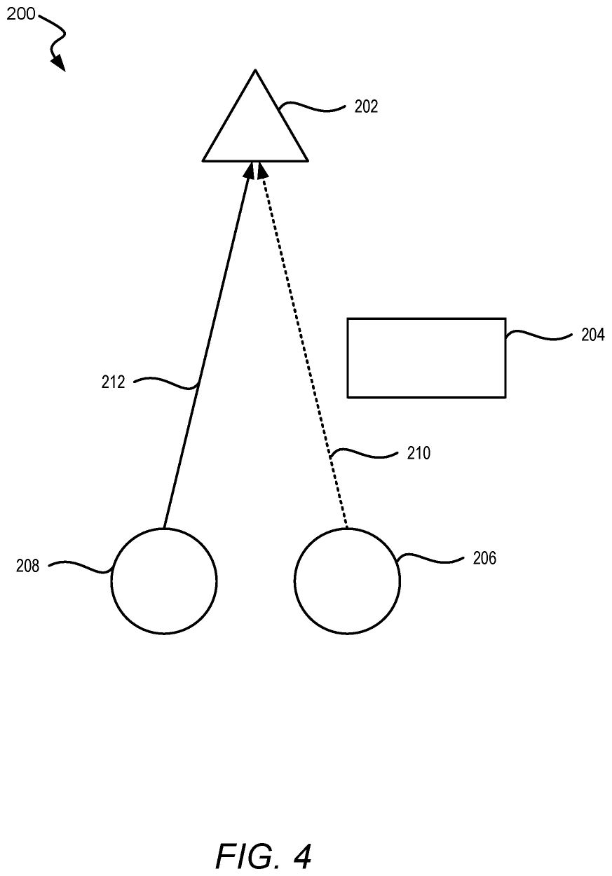

FIG. 4 shows the diagram 200 of both FIG. 2 and FIG. 3 with the first object 204 no longer obscuring the first gaze ray 210. FIG. 4 may represent a status of the system after the user has moved to the left or after the first object 204 has moved towards the right. The second gaze ray 212 is still maintained as the gaze ray used for ray-casting to select an object even though the first gaze ray 210 is no longer obstructed by the first object 204.

The system may maintain the eye dominance at the left eye 208 for a predetermined period of time, such as s few seconds, after a switch in eye dominance before reverting to the right eye 206 as the dominant eye as before. The reasoning behind maintaining the eye dominance at the left eye during a predetermined time period even when the right eye is no longer obstructed, is that it takes a while for the brain of a user to adapt to that the regularly dominant eye is no longer obstructed. Hence, the choice of gaze origin/dominant eye for ray-casting selection aims at simulating the behavior of the brain of the user, in order to provide an improved user experience.

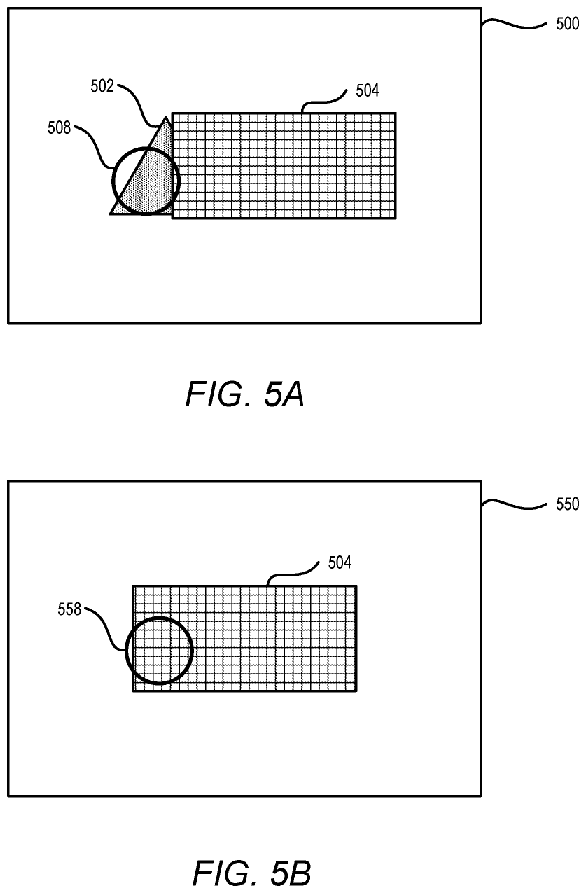

FIG. 5A and FIG. 5B shows a left eye view 500 and a right eye view 550 respectively, as perceived by a user of a second object 502 and a first object 504, according to at least one example. The views of FIG. 5A and FIG. 5B may represent the views of the left eye 208 and the right eye 206 respectively, of FIGS. 2-4. In FIG. 5A, the left eye view 500 includes a second object 502 further away in the field of view and a first object 504 nearer to the user than the second object 502. The left eye gaze area 508 is shown as a circular area including the location of intersection of the gaze ray from the left eye with the objects within the left eye view 500. As described above with respect to FIG. 3, the second object 502 is further away and partially obscured by the first object 504.

FIG. 5B shows the right eye view 550, showing the first object 504 obscuring the second object 502. Furthermore, the right eye gaze area 558, indicating where the gaze ray of the right eye intersects the first object 504 rather than reaching the second object 502 which is the intended focal point of the user.

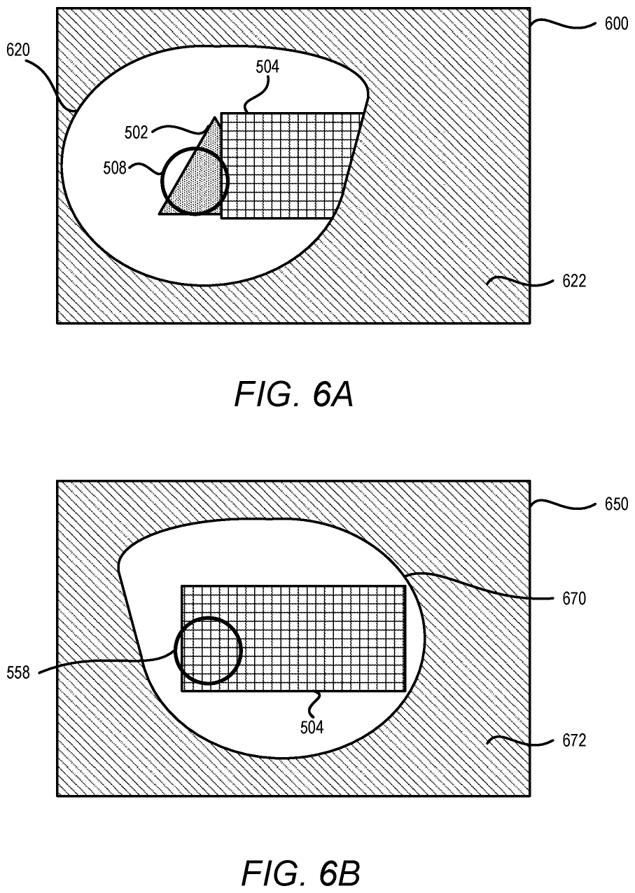

FIGS. 6A and 6B show the left eye view 600 and the right eye view 650 as perceived by the user, in one example, of the field of view shown in the diagram 200 of FIG. 3 and depicting the views of FIGS. 5A and 5B. Specifically, the views displayed include a first mask 622 and a second mask 672 which restrict the left eye view 600 and the right eye view 650 respectively. The first mask 622 and the second mask 672 block out or limit a digital field of view or landscape based on physical objects, such as the frame of a VR or AR headset which blocks the sightlines of the left eye and right eye of the user. The first profile 620 and the second profile 670 of the first mask 622 and the second mask 672 may correspond to the frames of the headset or may, in some examples, only limit some portions of the field of view. For instance, the first profile 620 may only block out a single box or bar where a physical portion of the headset interferes with the view of the user.

FIG. 7 illustrates an example flow diagram showing a process 700 according to at least one example. This process 700, and any other processes described herein, are illustrated as logical flow diagrams, each operation of which represents a sequence of operations that can be implemented in hardware, computer instructions, or a combination thereof. In the context of computer instructions, the operations may represent processor-executable instructions stored on one or more non-transitory computer-readable storage media that, when executed by one or more processors, perform the recited operations. Generally, processor-executable instructions include routines, programs, objects, components, data structures and the like that perform particular functions or implement particular data types. The order in which the operations are described is not intended to be construed as a limitation, and any number of the described operations can be combined in any order and/or in parallel to implement the processes.

Additionally, some, any, or all of the processes described herein may be performed under the control of one or more computer systems configured with specific executable instructions and may be implemented as code (e.g., executable instructions, one or more computer programs, or one or more applications) executing collectively on one or more processors, by hardware, or combinations thereof. As noted above, the code may be stored on a non-transitory computer readable storage medium, for example, in the form of a computer program including a plurality of instructions executable by one or more processors.

Turning now to FIG. 7, FIG. 7 illustrates an example flow chart depicting a process 700 for object selection based on ray-casting from dynamically shifting dominant eyes. The process 700 is performed by a computing device and may, in some case, also be performed by multiple computing devices.

The process 700 begins at block 702 by a computing device receiving position information of the first object 204 and the second object 202 displayed on a display device, wherein at least a part of the first object 204 is at a first depth and at least a part of the second object 202 is at a second depth. The depth of the first and second objects 204, 202, respectively, may be obtained by a forward facing depth sensor. In one example, the position information is part of a depth map corresponding to a field of view of a user. The field of view may correspond to a virtual environment, a physical environment, or a combination of both in AR. The depth map may be virtual and represent the depths of various objects within the field of view of the user as determined by a computing device or may also include inputs such as from the forward facing depth sensor which records distances to objects for an AR system. The depth sensor may be any suitable type of depth sensor such as a time of flight sensor, an infrared sensor, optical sensor and emitter, or any other suitable depth sensing device.

At block 704, the computing device receives eye tracking data from an eye tracking device indicating a first gaze ray and a second gaze ray. The first gaze ray may be the first gaze ray 210 and the second gaze ray may be the second gaze ray 212 of FIGS. 2-4. The first gaze ray indicates a gaze origin and a gaze direction from a first one of the two eyes and the second gaze ray indicates a gaze origin and a gaze direction from a second one of the two eyes.

At block 706, the computing device sets the first eye of the user as the dominant eye. In some systems this setting may be determined by the computing device recording a history of eye dominance and setting the dominant eye based on the eye which is more often the user's dominant eye. In some instances the user may input a setting into the computing device indicating that the first eye is the dominant eye based on their physical eye dominance.

At blocks 708 and 710, the computing device determines that the first gaze ray intersects a first object within the field of view of the user and that the second gaze ray intersects a second object. The first gaze ray originates at the first eye of the user and the direction of the ray is along the axis of the gaze direction indicated by the eye tracking device. The second gaze ray originates at the second eye of the user and the direction of the ray is along the axis of the gaze direction of the second eye. Any suitable eye tracking device may be implemented with the methods described herein, so long as the eye tracking device provides information on each of the eyes of the user independently.

As shown in FIG. 3, the first object and the second object may be different objects, while in some instances the first object and the second object may be the same object. For instance, in FIGS. 2 and 4, the first and second objects are the same object. In some examples the selection of different objects by each gaze ray may trigger the system to perform the methods described herein to accurately select the object within the environment that the user is focusing on.

At block 712, the process 700 includes the computing device determining a convergence depth of the first gaze ray and the second gaze ray. A number of methods may be implemented to determine the convergence depth of the first gaze ray and the second gaze ray. One such example is described above with respect to FIG. 2. The convergence depth represents a depth within the field of view where the gaze ray of the first eye and the gaze ray of the second eye meet to focus on a single area or object.

At block 714, the process 700 includes the computing device determining a first offset representing a difference between the depth of the first object and the convergence depth. The depth of the first object is based on the data from the position information, i.e. the depth sensor or the depth map, and the convergence depth is determined previously in block 712.

At block 716, the computing device determines a second offset representing a difference between the depth of the second object and the convergence depth. In a case where the first object and the second object are the same object, the first offset and the second offset may be identical.

At block 718, the computing device determines that the second offset is smaller than the first offset. The second offset being smaller than the first offset corresponds to the second object being at a depth within the field of view nearer to the convergence depth than the first object. The first object is therefore similar to the first object 204 of FIG. 3 in blocking the view of the second object 202 by the first eye.

In some examples, the first offset and the second offset may also both include an additional predetermined amount. For instance, the predetermined amount may represent an error value of the position information, including the depth map and/or the depth sensor, or the convergence depth. For instance, the first offset and the second offset may be near each other but not match the convergence depth. However, the predetermined amount may account for the errors in the measurements or dimensions and therefore not change or adjust the eye dominance unless the difference between the first offset and the second offset is larger than a trivial amount or a depth difference threshold. The depth difference threshold is a predetermined threshold amount set to account for errors in depth measurement from each of the eyes. In some instances the predetermined amount may be an absolute measurement of depth, while in some examples it may be a percentage of the depth, either to the first or second object or the convergence depth. Additionally, the predetermined amount may be different for the first offset and the second offset. For example, the second offset may include a second error which is larger than a first error of the first offset. This may be due to differences in the depth of the first and the second object or may be due to the quality of the data from the eye tracking device. For instance the data from the first eye may be more complete or accurate than the data from the second eye due to interference or other occlusions on the eye of the user.

The second offset may be less than a predetermined offset threshold, indicating that the second object is at or within a small deviation of the convergence depth. In such a case, the second eye may be set as the dominant eye based on the second offset being below the offset threshold. In an example, the predetermined offset threshold is set as the predetermined amount described above. In other examples, the predetermined offset threshold may be smaller or larger than the predetermined amount described above.

At block 720, the computing device sets the second eye as the dominant eye. This may include setting the eye dominance within the settings of the application or device running the eye tracking program. The second eye being set as the dominant eye may also include the computing device weighing a history of the second eye being the dominant eye. In addition to setting the second eye as the dominant eye, the process 700 may further include selecting an object based on the second gaze ray of the second eye as the dominant eye. The object selection uses the gaze ray of the dominant eye to identify the object the user is focusing on.

Setting the dominant eye includes determining a gaze origin of the gaze ray used for an eye tracking application. For instance, the eye tracking application may use the gaze origin for object selection. The gaze origin is placed at the dominant eye with the gaze ray matching the gaze direction of the eye set as dominant.

The second eye may be maintained as the dominant eye following the step of block 720 described above for a predetermined period of time, or until the process 700 is repeated to set the first eye as the dominant eye once again. The predetermined period of time may be a few seconds or may be a matter of minutes before reverting to the setting of the first eye as the dominant eye for object selection purposes. Furthermore, the process 700 may be repeated when the first offset and the second offset differ by more than an offset difference threshold, which may be selected or determined as described above with respect to the offset threshold.

In some examples, the process 700 may further include updating the position information and/or the depth map after setting the second eye as the dominant eye, the update to the position information and/or the depth map including changes to the environment as the user moves. The second eye may be retained as the dominant eye following the update to the position information and/or the depth map for a predetermined period of time as described above.

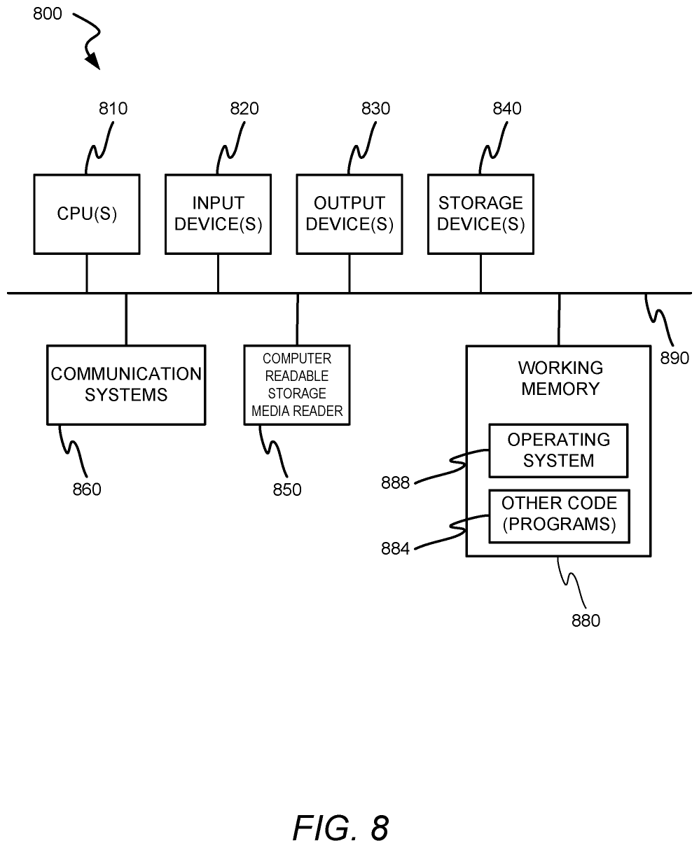

FIG. 8 is a block diagram illustrating a computer system 800 in which embodiments presented herein may be implemented. This example illustrates a computer system 800 such as may be used, in whole, in part, or with various modifications, to provide the functions of the eye tracking device 120, the graphics processing device 130, the game console, the computing device 140, and/or other components discussed above. For example, various functions of the eye tracking device 120 and associated processors may be controlled by the computer system 800, including, merely by way of example, tracking a user's gaze point, determining an anticipated gaze point, controlling the graphics processing device 130, etc.

The computer system 800 is shown comprising hardware elements that may be electrically coupled via a bus 890. The hardware elements may include one or more central processing units 810, one or more input devices 820 (e.g., a mouse, a keyboard, etc.), and one or more output devices 830 (e.g., a display device, a printer, etc.). The computer system 800 may also include one or more storage device(s) 840. By way of example, storage device(s) 840 may be disk drives, optical storage devices, a solid-state storage device such as a random access memory (“RAM”) and/or a read-only memory (“ROM”), which can be programmable, flash-updateable and/or the like.

The computer system 800 may additionally include a computer-readable storage media reader 850, a communications system 860 (e.g., a modem, a network card (wireless or wired), an infra-red communication device, Bluetooth™ device, cellular communication device, etc.), and working memory 880, which may include RAM and ROM devices as described above.

The computer-readable storage media reader 850 can further be connected to a computer-readable storage medium, together (and, optionally, in combination with storage device(s) 840) comprehensively representing remote, local, fixed, and/or removable storage devices plus storage media for temporarily and/or more permanently containing computer-readable information. The communications system 860 may permit data to be exchanged with a network, system, computer and/or other component described above.

The computer system 800 may also comprise software elements, shown as being currently located within a working memory 880, including an operating system 884 and/or other code 888. It should be appreciated that alternate embodiments of a computer system 800 may have numerous variations from that described above. For example, customized hardware might also be used and/or particular elements might be implemented in hardware, software (including portable software, such as applets), or both. Furthermore, connection to other computing devices such as network input/output and data acquisition devices may also occur.

Software of computer system 800 may include code 888 for implementing any or all of the function of the various elements of the architecture as described herein. For example, software, stored on and/or executed by a computer system such as computer system 800, can provide the functions of the eye tracking device 120, the graphics processing device 130, the game console, the processor/computer, and/or other components such as those discussed above. Methods implementable by software on some of these components have been discussed above in more detail.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, it should be understood that the present disclosure has been presented for purposes of example rather than limitation, and does not preclude inclusion of such modifications, variations, and/or additions to the present subject matter as would be readily apparent to one of ordinary skill in the art. Indeed, the methods and systems described herein may be embodied in a variety of other forms; furthermore, various omissions, substitutions and changes in the form of the methods and systems described herein may be made without departing from the scope of the present disclosure. The accompanying claims and their equivalents are intended to cover such forms or modifications as would fall within the scope of the present disclosure.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provide a result conditioned on one or more inputs. Suitable computing devices include multipurpose microprocessor-based computing systems accessing stored software that programs or configures the computing system from a general purpose computing apparatus to a specialized computing apparatus implementing one or more embodiments of the present subject matter. Any suitable programming, scripting, or other type of language or combinations of languages may be used to implement the teachings contained herein in software to be used in programming or configuring a computing device.

Conditional language used herein, such as, among others, “can,” “could,” “might,” “may,” “e.g.,” and the like, unless specifically stated otherwise, or otherwise understood within the context as used, is generally intended to convey that certain examples include, while other examples do not include, certain features, elements, and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Similarly, the use of “based at least in part on” is meant to be open and inclusive, in that a process, step, calculation, or other action “based at least in part on” one or more recited conditions or values may, in practice, be based on additional conditions or values beyond those recited. Headings, lists, and numbering included herein are for ease of explanation only and are not meant to be limiting.

The various features and processes described above may be used independently of one another, or may be combined in various ways. All possible combinations and sub-combinations are intended to fall within the scope of the present disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed examples. Similarly, the example systems and components described herein may be configured differently than described. For example, elements may be added to, removed from, or rearranged compared to the disclosed examples.

What is claimed is:

1. A method in an eye tracking system for gaze origin selection based on dominant eye shift of a user, comprising:
receiving position information of a first object and a second object displayed on a display device;
receiving eye tracking data indicating a first gaze ray of a first eye of the user and a second gaze ray of a second eye of the user;
setting the first eye as a dominant eye;
determining that the first gaze ray intersects the first object at a first depth;
determining that the second gaze ray intersects the second object at a second depth;
determining a convergence depth at a convergence of the first gaze ray and the second gaze ray;
determining a first offset between the convergence depth and the first depth;
determining a second offset between the convergence depth and the second depth;
determining that the second offset is smaller than the first offset; and
setting the second eye as the dominant eye based on the second offset being smaller than the first offset.

2. The method of claim 1, further comprising:
selecting the second object based on the second gaze ray after the second eye is set as the dominant eye.

3. The method of claim 1, wherein:
determining that the second offset is smaller than the first offset comprises determining the second offset plus a first predetermined amount is smaller than the first offset.

4. The method of claim 1, further comprising:
determining that a difference between the first offset and the second offset is larger than an offset difference threshold, wherein setting the second eye as the dominant eye, is further based on the difference being larger than the offset difference threshold.

5. The method of claim 1, wherein:
setting the first eye as the dominant eye, is based on a user input indicating that the first eye is the dominant eye.

6. The method of claim 1, further comprising:
maintaining the second eye as the dominant eye for a predefined time period.

7. The method of claim 1, wherein:
setting the first eye as the dominant eye is based on a history indicating that the first eye is the dominant eye based on previous user eye dominance determinations.

8. The method of claim 1, further comprising:
determining that the second offset is less than an offset threshold; and
wherein setting the second eye as the dominant eye is further based on the second offset being less than the offset threshold.

9. The method of claim 1, wherein the position information is part of a depth map, the method further comprising:
restricting the depth map based on physical objects obstructing the first gaze ray and the second gaze ray.

10. The method of claim 1, further comprising:
determining that a difference between the first depth and the second depth is larger than a depth difference threshold, wherein setting the second eye as the dominant eye is further based on determining that the difference between the first depth and the second depth is larger than the depth difference threshold.

11. A computer system, comprising:
a non-transitory computer-readable medium storing instructions; and
a processing device communicatively coupled to the non-transitory computer-readable medium, wherein the processing device is configured to execute the instructions and perform operations comprising:
receiving position information of a first object and a second object displayed on a display device;
receiving eye tracking data indicating a first gaze ray of a first eye of a user and a second gaze ray of a second eye of the user;
setting the first eye as a dominant eye;
determining that the first gaze ray intersects the first object at a first depth;
determining that the second gaze ray intersects the second object at a second depth;
determining a convergence depth at a convergence of the first gaze ray and the second gaze ray;
determining a first offset between the convergence depth and the first depth;
determining a second offset between the convergence depth and the second depth;
determining that the second offset is smaller than the first offset; and
setting the second eye as the dominant eye based on the second offset being smaller than the first offset.

12. The computer system of claim 11, wherein the operations further comprise:
selecting the second object based on the second gaze ray after the second eye is set as the dominant eye.

13. The computer system of claim 11, wherein:
determining that the second offset is smaller than the first offset comprises determining the second offset plus a first predetermined amount is smaller than the first offset.

14. The computer system of claim 13, wherein:
the first predetermined amount comprises a first error of the first offset and a second error of the second offset.

15. The computer system of claim 11, wherein:
setting the first eye as the dominant eye is based on a user input indicating that the first eye is the dominant eye.

16. The computer system of claim 11, wherein:
setting the first eye as the dominant eye is based on a history indicating that the first eye is the dominant eye based on previous user eye dominance determinations.

17. The computer system of claim 11, wherein the operations further comprise:
determining that a difference between the first depth and the second depth is larger than a depth difference threshold, wherein setting the second eye as the dominant eye is further based on determining that the difference between the first depth and the second depth is larger than the depth difference threshold.

18. The computer system of claim 11, wherein the position information is part of a depth map, and wherein the operations further comprise:
restricting the depth map based on physical object obstructing the first gaze ray and the second gaze ray.

19. The computer system of claim 11, further comprising:
determining that the second offset is less than an offset threshold; and
wherein setting the second eye as the dominant eye is further based on the second offset being less than the offset threshold.

20. One or more non-transitory computer-readable media storing instructions, that upon execution on a system, cause the system to perform operations comprising:
receiving position information of a first object and a second object displayed on a display device;
receiving eye tracking data indicating a first gaze ray of a first eye of a user and a second gaze ray of a second eye of the user;
setting the first eye as a dominant eye;
determining that the first gaze ray intersects the first object at a first depth;
determining that the second gaze ray intersects the second object at a second depth;
determining a convergence depth at a convergence of the first gaze ray and the second gaze ray;
determining a first offset between the convergence depth and the first depth;
determining a second offset between the convergence depth and the second depth;
determining that the second offset is smaller than the first offset; and
setting the second eye as the dominant eye based on the second offset being smaller than the first offset.
